(19) United States
(12) Patent Application Publication
(10) Pub. No.: US 2011/0009693 A1
(43) Pub. Date: Jan. 13, 2011

(54) METHOD AND DEVICE FOR INDUCING EROTIC STIMULATION WHILE ASLEEP

(76) Inventor: Daniel Westerband Borrero, (US)

Correspondence Address: JACQUELINE TADROS, P.A., 500 CYPRESS CREEK ROAD WEST, SUITE 350, FORT LAUDERDALE, FL, 33309, US

(21) Appl. No.: 12/885,458

(22) Filed: Sep. 18, 2010

Related U.S. Application Data
(63) Continuation-in-part of application No. 11/581,736, filed on Oct. 16, 2006.

(51) Int. Cl. A61F 5/00; A61H 1/00

(52) U.S. Cl. 600/38; 601/46

(57) ABSTRACT

A vibration device for inducing erotic stimulation at a pre-set time, including a housing, a power source for powering the vibration device; a timing unit; including timer control circuitry disposed within the housing and electrically coupled to the power source, an offset weight driven by a motor and powered by the powering source to generate vibrations and impart a vibratory motion to a region of the body at the pre-set time.

### CROSS-REFERENCE TO RELATED APPLICATIONS

We hereby claim benefit under Title 35, United States Code, Section 120 of U.S. patent application Ser. No. 11/581,736 filed on Oct. 16, 2006 (hereinafter “Prior Application”). This application is a Continuation-in-Part of the Prior application. The Prior application is currently pending. The Prior application is hereby incorporated in its entirety by reference into this application.

### FIELD OF THE INVENTION

The present invention relates generally to the field of vibrators and more particularly to a battery operated motorized vibrator for inducing sexual or erotic stimulation.

### BACKGROUND OF THE INVENTION

Vibratory devices, also known as massagers, vibrating massagers and numerous other names are well-known in the art. They come in a variety of configurations and perform a variety of functions, ranging from medical therapy to erotic or sexual stimulation. They may be battery powered or run on conventional alternating current electricity.

Vibratory devices are known for use in applying vibratory therapy to selected regions of the body. Such devices often include a motor driven vibrator unit for imparting a physically detectable vibratory motion to a selected region of the body. The use of vibratory devices to impart erotic and sexual stimulation has been the subject of innovation and design activity for numerous years. Over the last several decades, the commercial market has seen the introduction of many different types of motorized vibrators. The demand for these devices is substantial as is evidenced by a well established commercial industry.

Some known devices are shown in U.S. Pat. No. 7,081,087 naming as inventor, Jannuzzi, U.S. Pat. No. 6,350,230, naming as inventor, Kontos, and U.S. Pat. No. 5,573,499, naming as inventor McAllister. Each of these patents teaches a sexual aid device that utilizes a motorized vibrator for erotic and sexual stimulation.

These and other vibratory devices known in the art require the subject or another party to activate or manipulate the vibratory device at the time of initiating or inducing stimulation.

WebSciences International is dedicated to promoting research in all aspects of the neurosciences, with an emphasis on research that deals with sleep and wakefulness. WebSciences International was founded in 1992 to serve the biomedical and research communities. Research at WebSciences International is funded principally by grants from the National Institute of Health.

As per research conducted by WebSciences International, activation of the sexual organs during sleep is a commonplace occurrence in both males and females. In women, vaginal blood flow has been measured with a thermoconductance flow meter. In men, the most common measurement technique involves placing two small elastic circular mercury-filled capillary strain gauges around the penis, one at the base and one at the tip. The strain gauges measure the changes in penile diameter that occur with tumescence, defined herein as a readiness for sexual activity marked especially by vascular congestion of the sex organs. Both types of measures are recorded on a polysomnograph along with EEG, EOG, and EMG activity. By this means, changes in vaginal or in penile circumference can be correlated with different stages of sleep and wakefulness.

Episodes of tumescence are clearly associated with REM sleep periods, although tumescence is not exclusively confined to REM sleep. Studies that have been performed in males and females have found a relationship between sexual arousal and REM sleep.

While in a sleeping state, a subject is not able to activate or initiate a vibratory device to induce stimulation. Thus there exists a need for, and it would be beneficial and advantageous to have, a vibratory device to induce stimulation, including but not limited to erotic or sexual stimulation, on a subject at a future time, for example, during a period of sleep without the additional intervention of another party to activate or manipulate the vibratory device.

All patents and other publications cited herein are expressly incorporated by reference in their entirety.

### SUMMARY OF THE INVENTION

The present invention provides a method for inducing erotic or sexual stimulation in a subject at a future time and relates to further improvements in therapeutic vibratory devices to enhance the utility and soothing effects thereof.

The present invention offers an alternative to vibratory devices known in the art. The vibrator of the present invention incorporates a timing mechanism that allows a user to pre-set a time for activation of the vibratory device.

Thus the present invention provides a device and method for initiating or inducing erotic or sexual stimulation on a subject at a pre-selected time. The timing mechanism of the present invention also allows a user to select and pre-set the duration of the vibratory stimulation. Thus, an advantage of the present invention is that a user may set the vibratory device for activation at a future time, for example, during a period of sleep.

In accordance with one embodiment of the present invention, the dimensions of the device are miniature, that is generally two to three inches in length and one half to one inches wide, so as to allow for complete and facile insertion of the device in the vaginal region in a manner that does not cause discomfort or hinder a subject's ability to fall asleep.

Another advantage of the present invention is that a subject may induce stimulation of the Grafenberg (“G”) spot while the subject is asleep, by setting the timer to activate the device to a time when the subject will be asleep and setting the timer to deactivate the device at a time when the subject is expected to still be in asleep, then inserting the device completely within the vaginal region prior to falling asleep.

Still another advantage of the present invention is that it can induce erotic stimulation during a period of sleep without the additional intervention of another party to activate or manipulate the vibratory device.

An object of the present invention is to induce pleasurable or erotic stimulation on a subject during a period of sleep, without the additional intervention of another party, to coincide with when studies show that both males and females are predisposed to activation of the sexual organs,

Another aspect of the present invention is a removable sleeve that encases the vibratory device and is made of silicon or other material pleasing to the touch. In addition to providing the user with a more pleasing sensation, the removable sleeve will help to preserve the housing of the vibratory device and foster hygiene.

In accordance with one embodiment of the present invention, the vibratory device is surrounded and enclosed in a cushioning element, including but not limited to a gelatinous elastomer.

An object of the invention is to utilize a cushioning element that conforms to the shape of the vibratory device while generally evenly distributing a supporting force across the contact area of the cushioned object and avoiding pressure peaks to impart a greater sense of comfort and a pleasurable sensation on a selected region of the body.

Another object of the present invention is to utilize a vibratory device on an external region of the body.

In another embodiment of the present invention, the vibratory device is scaled to a length of approximately between three and four inches and a diameter of approximately between five-eighth and one inch to allow for minimally invasive internal use of the vibratory device.

Thus the vibratory device of the present invention provides numerous advantages, including but not limited to enabling a subject to partake in auto-erotic stimulation while in a sleeping or restful state. Once the timing mechanism of the vibratory device of the present invention is set, no additional action or manipulation of the vibratory device is required.

The present invention as described herein incorporates advantages of other vibratory devices known in the art, as well as other novel features, resulting in a method and device for inducing stimulation which is new, not anticipated, rendered obvious, suggested or implied by any of the prior art, either alone or in any combination thereof.

Other objects and advantages of the present invention will become obvious to the reader and it is intended that these objects and advantages are within the scope of the present invention.

There has thus been outlined, rather broadly, the more important features of the invention in order that the detailed description thereof may be better understood, and in order that the present contribution to the art may be better appreciated. Other features and advantages of the present invention will become apparent from the following more detailed description, taken in conjunction with the accompanying drawings, which illustrate, by way of example, the principles of the invention.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

FIG. 1 is a cross sectional view of a vibratory device of the present invention, taken generally along lines 1-1 of FIG. 4, illustrated without a protective sleeve.

FIG. 2 is a top plan view of a preferred embodiment of the invention having a gel pack.

FIG. 3 is a cross-sectional view of an alternate embodiment of the vibratory device of the present invention, taken generally along lines 1-1 of FIG. 4, and illustrating a unitary enclosure with a battery cap, having on/off and timer buttons and a timer screen having an alpha-numeric display on front surface of the housing, shown with the protective sleeve.

FIG. 4 is a perspective view of the invention, illustrating the protective sleeve covering the housing.

### DETAILED DESCRIPTION OF THE INVENTION

The subject invention is illustrated in the attached drawings referred to herein. Throughout the drawings and the description below, the same reference numerals will be used to identify identical elements.

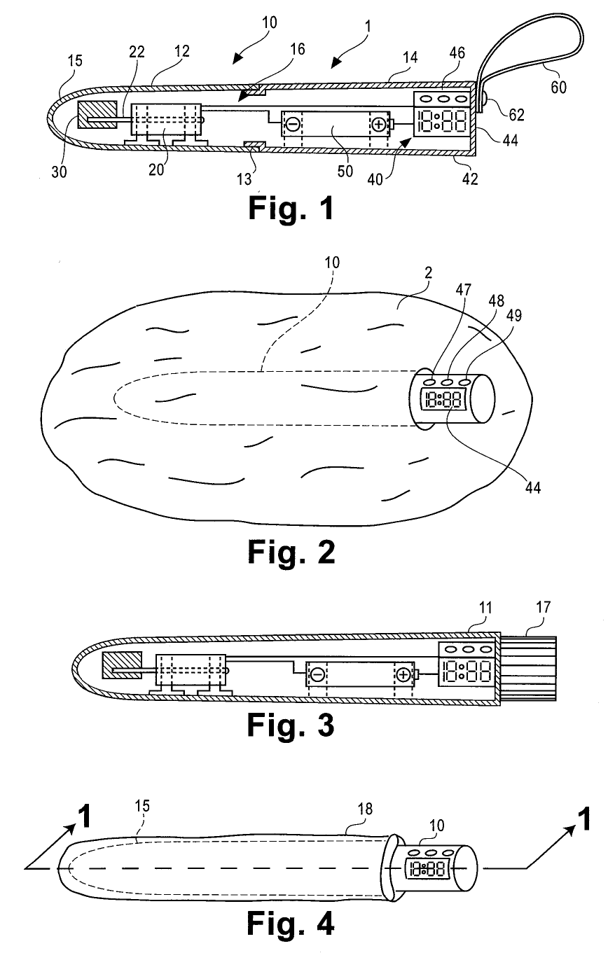

As shown in FIG. 1, a vibratory device 1 includes a housing 10, a DC motor 20, an offset weight 30, a timer 40, a DC power source 50 and a flexible strip 60.

The housing 10 of the vibratory device 1 includes mating members 12 and 14 and a hollow region 16. Mating members 12, 14 are further defined to include a lower member 12, and an upper member 14. The upper member 12 is removably secured to the lower member 14 by snap fitting or other fastening means 13 as known in the art.

The housing 10 is substantially cylindrical and generally elongated with a rounded anterior portion to facilitate insertion of the vibratory device. The timer 40 is positioned on a distal end of the housing 10.

The housing 10 is formed of a semi-rigid material, for example, a durable yet moldable plastic material as is known in the art, and then sheathed in a removable close fitted sleeve or mold 18 as shown in FIG. 4. The sleeve 18 fits snugly over the housing 10 and is made of silicon, rubber or other like material having a pleasing texture. In this embodiment, the vibration device 1 sheathed with the sleeve 18 is for use on an internal region of a body.

FIG. 3 illustrates another embodiment of the present invention. In this embodiment, the housing 10 is a substantially unitary member 11 and is generally elongated with a preferably rounded or tapered first end 15. A second end preferably includes a detachable cap 17 for accessing the interior cavity of the housing 10. Referring to FIG. 1, the housing 10 of the vibratory device 1 is shown to contain the DC motor 20, the offset weight 30, the timer 40 and the DC power source 50. The power source 50 may include, but is not limited to, one or more typical household batteries, and is in electrical communication with the motor 20 for powering the motor 20. The power source 50 is removably secured within a portion of the lower shell 12 by means of a conventional mounting bracket (not shown).

The motor 20 is supported with a typical motor mount (not shown) which is in contact with the outside surface of the motor 20 and the inside surface of the lower shell 12 of the housing 10, as is well-known in the art.

The DC motor 20 is provided with a driven shaft 22 on which the offset weight 30 is secured for rotation. Thus the offset weight 30 and DC motor 20 are attached in mechanical communication. The result is vibratory displacement of the offset weight 30 when the motor 20 is activated by the power source 50. As the motor 20 rotates, the offset weight 30 causes the motor 20 to vibrate. As the motor 20 vibrates, the vibration passes from the motor 20 to the housing 10 and is transmitted to a region of the body. All electrical components including the power source 50 and its related connectors, motor 20 and wiring are sufficiently protected with sealants and gaskets (not shown) to prevent wetting from external sources, including liquids or gels. Other motor arrangements may be used and still remain within the scope of the invention.

The timer 40 is provided to control operation of the vibratory device 1 and is in electrical communication to the power source 50 and the motor 20. The timer includes a timer control circuitry 42. The control circuitry's 42 power is received from the DC power source 50 disposed within the housing 10. The timing control circuitry 42 includes a printed circuit board (not shown) disposed inside the housing 10 for time presetting and computing.

Referring now to FIG. 3, a set of setting buttons 46 and a timer screen 44 are positioned on the front surface of the housing 10. The timer screen 44 is preferably a liquid crystal display having appropriate alphanumeric displays to indicate the current time, the pre-set on time and the pre-set off time.

The setting buttons 46 include a timer on/off button 47, an hour button 48 and a minute button 49. The set of setting buttons 46 are electrically connected to the timer control circuitry 42.

The setting buttons 46 may be used to enter a pre-set on or off time. Referring to FIGS. 1-4, the timing control circuitry 42 is electrically coupled to the power source 50 and the DC motor 20. When a pre-set on time is entered, the timer control circuitry 42 generates an electrical signal to activate the motor 20. When a pre-set off time is entered, the timer control circuitry 42 generates a disconnecting signal to de-activate the motor 20.

Thus, the timer control circuitry 42 generates a signal to activate the power source 50. Hence, by setting the timer 40 to open a DC current to the motor 20 at a pre-selected time, an electrical connection between the power source 50 and the motor 20 is established. Similarly, the timer control circuitry 42 generates a disconnecting signal to close the DC current to the motor 20 upon a pre-selected time thereby deactivating the motor 20 and the vibratory device 1. The pre-set off-time may be configured either as an absolute off-time, or as an elapsed time from the pre-set on-time. For example, if the pre-set on time is 4:00 am, then the pre-set off time may be set as either a duration of time that the device 1 is activated (such as five minutes), or as an absolute off-time, such as 1:05 am.

The DC power source 50 is disposed within the housing 10 and provides electrical power for the timer control circuitry 42. The action of the DC motor 20 is controlled by the timer control circuitry 42 to which the motor 20 is electrically connected.

FIG. 2 illustrates another embodiment of the vibratory device 1. In this embodiment, the vibratory device 1 is encased within a cushioning medium 2. The preferred cushioning medium 2 is a gelatinous elastomer, gelatinous viscoelastomer or a gel pack as is known in the art. The cushioning medium 2 is preferably tubular and fits over and around the vibratory device. However, it is appreciated that various configurations of cushioning elements may be used without departing from the spirit or scope of the invention.

In this embodiment, the vibratory device 1 is encased within the cushioning medium 2 for placement on an external region of the body to offer the subject an alternative means of inducing stimulation. In a preferred embodiment, the removable sleeve 18 fits over the cushioning medium 2.

A cord 60 is removably connected to the housing 10 by a fastening means 62 as is known in the art, for example a hook. The cord 60 provides a convenient handle for facilitating the insertion and extraction of the device 1 from the vaginal cavity.

While there has been shown and described the preferred embodiments of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

I claim:

1. A vibratory device for inducing erotic vaginal stimulation to a subject while in a sleeping state, comprising:
a substantially cylindrical and generally elongated housing having a generally rounded anterior portion, the housing including a lower and upper member, wherein said upper member is removably secured to said lower member, and wherein said lower and upper member define a hollow region when said upper and lower member are secured in a fixed mating arrangement;
a motor having a motor mount, wherein said motor mount is in proximate contact with an inside surface of said lower member, the motor further including a shaft on which an offset weight is secured in mechanical communication for rotation;
a power source, wherein said power source is removably secured within the lower member of said housing and wherein said power source is in electrical communication with said motor for powering said motor, resulting in a vibratory displacement of said offset weight and vibration of said motor; and
a timer, including a timer control circuitry, in electrical communication to said power source and motor, for controlling the operation of said vibratory device.

2. The device of claim 1, further including means removably secured to said housing for handling the vibratory device.

3. The device of claim 1, wherein said housing is formed of a semi-rigid moldable plastic material.

4. The device of claim 1, wherein said housing is cased in a removable close fitted sleeve.

5. The device of claim 4, wherein said removable sleeve comprises a silicone rubber material.

6. The device of claim 1, wherein said upper member is removably secured to said lower member by snap fitting means.

7. The device of claim 2, wherein said means is a cord for facilitating the insertion and extraction of said device.

8. The device of claim 1, wherein said housing includes a distal end for sheltering said timer.

9. The device of claim 1, wherein said power source includes at least one battery.

10. The device of claim 1, wherein said power source is removably secured within said lower member by means of a conventional mounting bracket.

11. The device of claim 3, wherein a generally tubular cushioning medium encases the device for placement of the device on an external region of a body.

12. The device of claim 11, wherein the cushioning medium is a gel pack.

13. The device of claim 1, wherein said housing is sized to allow for complete, smooth and facile vaginal insertion of the entire device, such that the completely inserted device is tolerated in comfort and does not hinder the subject from falling asleep.

14. A method of inducing vaginal erotic stimulation to a subject while in a sleeping state comprising the steps of:
providing a device for inducing erotic stimulation comprising a substantially cylindrical and generally elongated housing having a generally rounded anterior portion, the housing including a lower and upper member, wherein said upper member is removably secured to said lower member, and wherein said lower and upper member define a hollow region when said upper and lower member are secured in a fixed mating arrangement; a motor having a motor mount, wherein said motor mount is in proximate contact with an inside surface of said lower member, the motor further including a shaft on which an offset weight is secured in mechanical communication for rotation; a power source, wherein said power source is removably secured within the lower member of said housing and wherein said power source is in electrical communication with said motor for powering said motor, resulting in a vibratory displacement of said offset weight and vibration of said motor; and a timer, including a timer control circuitry, in electrical communication to said power source and motor, for controlling the operation of said vibratory device;
entering a pre-set activation time into said timing unit for activation when the subject is asleep; and
entering a pre-set deactivation time to generate a deactivating signal at said pre-set off time while the subject is asleep; and
inserting the device completely within said subject's vaginal cavity.
